(19) United States
(12) Patent Application Publication
(10) Pub. No.: US 2018/0338982 A1
(43) Pub. Date: Nov. 29, 2018

(54) Cooperative Medication Combination Systems

(71) Applicant: Kevin Pickering, Peoria, AZ (US)

(72) Inventor: Kevin Pickering, Peoria, AZ (US)

(21) Appl. No.: 15/607,441

(22) Filed: May 27, 2017

(51) Int. Cl. A61K 31/55; A61K 31/403; A61K 31/401; A61K 31/4035; A61K 31/4422; A61K 31/4168; A61K 31/502; A61K 31/155; A61K 31/4985; A61K 31/18; A61K 31/4439; A61K 31/366; A61K 31/216; A61K 31/397; A61K 31/135; A61K 31/137; A61K 31/496; G06F 19/00

(52) U.S. Cl. CPC A61K 31/55; G06F 19/325; A61K 31/401; A61K 31/4035; A61K 31/4422; A61K 31/4168; A61K 31/502; A61K 31/155; A61K 31/4985; A61K 31/18; A61K 31/4439; A61K 31/366; A61K 31/216; A61K 31/397; A61K 31/135; A61K 31/137; A61K 31/496; G06F 19/3456; A61K 31/403

(57) ABSTRACT

This invention captures and teaches the high-level method of combining doses of medications in unconventionally substandard amounts for the cooperative treatment of medical pathologies with the end product of a pharmaceutical composition. By combining multiple medications, each treating the same disease process and each in a calculated substandard dosage, it is rational to allow for greater comprehensive efficacy while simultaneously bypassing conventional side-effects, clinically significant medication interactions, and other potentially unforeseen deleterious effects, all because the dosage composition is small enough individually and collaborative chemical diversity manifests favorable kinetic dynamics, thereby mitigating unwanted drug effects while enhancing the targeted indication.

### CROSS-REFERENCE TO RELATED APPLICATIONS

61/499,060, Jun. 20, 2011, Cooperative Drug Combination Systems, same inventor's unintentional provisional application abandonment via patent lawyer discrepancy

Ser. No. 14/048,694, Continuation In Part Ser. No. 14/965,912, Dec. 11, 2015, Cooperative Medication Combination Systems, likewise same inventor's application USPTO did not file further continuation application.

### BACKGROUND FOR THE INVENTION

Throughout history, art is judged upon the ancient Socratic notion of the techne of measure, a weighing of the intellectual good versus bad. Teaching such art toggles from the abstract to the empirical. In the case of medicine, the pleasure versus pain, the practical aspect of good and bad, is expounded upon, from a health perspective, by weighing therapy versus deleterious side-effects in relation to a given malady.

Therapy in this sense would be called medicine. In the case of this patent text: therapy, such as medicine, a drug, chemical agent, chemical compound, pharmaceutical medication, prescription medication, etc. will be synonymous with a pharmaceutical composition. Emphasising the importance of therapy with pharmaceutical compositions is related to standard doses.

A malady is also considered an illness, disease, disorder, syndrome, condition, sickness or even a pathology. Much of this application teaching focuses upon the high level utility of the application invention, as the very utility of the pharmaceutical composition use process is the purpose of this improved method of improving disease therapy. Further importance of pharmaceutical composition therapy is related to standard indications in addition to standard doses.

Standard doses for standard indications make the method of combination, via algorithm, clear and distinct. This algorithm begets further treatment algorithms that make the method of cooperative medication combination systems instill exponential improvement in disease state management, via pharmaceutical composition technology.

With the current conventional pharmaceutical medical model, a single chemical agent is often sought, with extensive resources, to counteract a given pathology, with the intent of an all-encompassing cure, prevention of pathology, or total illness symptom alleviation. The current conventional medical model, via one compound, may individually possess multiple potent mechanisms, but one chemical compound nonetheless. At times, other potent compounds are added to a final composition to further the end desired effect of enhancing one ultimate targeted endpoint, achieving an indication without auxiliary negative effects. The extensive resources invested in this current singularly potent chemical compound model have provided many instances of novel mechanisms to accomplish similar therapeutic goals.

The current medical model can given the appearance there are only singularly distinct methods to treat distinct disease, much less a tangible method of significant similarity connecting such well defined treatments with well defined diseases, standardized doses with standardized indications. In other words, it may appear the method making, treating or even dosing diseases such as common illnesses of uncomplicated hypertension, diabetes mellitus, dyslipidemia, symptoms of depression, etc. are so distinct they have no practical utility related to each other. This patent aims to demonstrate there is a striking need regarding the method to treat various diseases, and a striking need related to the method to make various pharmaceutical compositions for various diseases, a better disease based method. This related need is by means of the method related to dose various pharmaceutical compositions treating various diseases. In fact, this patent teaches the similarity of various diseases and various pharmaceutical compositions by demonstrating a method of combining various substandard doses of pharmaceutical compositions to treat disease, albeit at a highly advanced level.

Ultimately, this patent instills active practical steps to improve the rational similarity behind the current method of treating disease with pharmaceutical compositions. In so doing, the alteration of the dosing and consequent cooperative combinations of disease specific standard classes of medication are contoured with an algorithm that best treats disease with the available pharmaceutical composition technology.

Perhaps all can compare this method rationality to the process of rearing children. Since all have familiarity with such a familial dynamic, one need not be a polymath of therapeutics to appreciate such a comparison. Therapeutic treatment can be akin to parenting: rearing in general, rearing over time, rearing within the same family, etc. Distinct techniques of improved parenting exist, with distinct pitfalls. No technique has been proven best, but the timing of applying any technique appears critical. The quantity of any one technique application is also crucial. Techniques are found at each level of the rearing process. The quicker the appropriate technique is used, the better the outcomes. This method is a form of combining practical techniques, at a high level, appreciating the timing, quantity, and need for enhanced technology. Medicine can be easier, as the quantification aspect is of greater distinct convention than rearing children.

Readily achieving the desired protoplasmic balance requires a revolutionary process approach. The pharmaceutical market is flooded with the current fractionated therapeutic model of particular mechanisms seemingly not being enhanced to therapeutic potential. With these novel mechanisms of pharmaceutical chemical therapy have come novel side-effects, often unwanted side-effects. This current gold standard of conventional medication research and development has provided many redundant duplicates of clinical species, some of which are bereft of creative utility and whose existence hinges upon the success of marketing and lesser standards. Consequently, there is a glut of fractionated instances of attempting to treat the totality of a given complex pathological condition, but no enhanced method to better capture a greater potential of the currently available pharmaceutical technology. The landmark cooperative medication combination therapy method stands as a revolutionary model, a closer attempt to lessen pathology by increasing health and minimizing side-effects.

It is often accepted that most initial teachings regarding abstract concepts and/or high level concepts with many moving parts need to be simplified. The human body is often simplified for educational reasons. Likewise, pharmaceutical medications are simplified for similar inculcation. When the human body is attempted to be understood in relation to medication, then the oversimplifications can become markedly excessive. The lack of fully understanding the relationship of medicine and the human body has been further disconnected through profit marketing for disease treatment, leaving a significant reality gap between the deeply intricate balance within the complexities of human medicine.

It is assumed the history of medicine began when it was noted that people become sick. Currently, conventional medicinal science is often a race to uncover the better appreciated molecular nature of disease, thereby revealing a medicinal code to reverse, prevent or lessen disease from such a deciphered grail. It would seem that the single potent mechanistic pharmaceutical compound treating a corresponding disease was at one point adequate, at least the best offered at the time. Then disease knowledge appeared to became more advanced. Simultaneously, disease treatment became more complex. Critiques of both became more widespread, some gaining legitimacy. Perhaps the most damaging critique of modern medicinal knowledge is that profit has remained profit, but the fetish with such, as noted by keen social scientists, has appeared to heighten to such a degree that therapeutic sensibilities have become more significantly distorted along the way.

Within the social system of medicine, the notoriety, be it corporate or individual, has encouraged a seemingly wasteful and disconnected system. Companies are encouraged to keep health research information secretive so as to not impede the ultimate short-term corporate agenda. Researchers are educated in a system that requires such a philosophical doctor to dissect and articulate a reality none have ever intellectually unraveled. These terminal recapitulations isolate and force a sense of disconnect, be it in a cubicle, lab, a hospital bed, or otherwise isolated realm. This paradigm can overlook what should be clear, or find incentives beyond the common good to disenchant a sustainable idea, an idea guided by a genuine sense of bettering humanity with available resource processes.

Current medical convention views the molecular level of the human body through microscopic receptors. In a physical sense, humans are a composition of various cells, cells that can be differentiated with a lifetime of minutia. Medications generally bring about a change in the human body through reactions via various cellular receptors. The receptors in turn will generate or disable a series of chemical events in the body. Some medications affect multiple receptors. Modern medicine generally aspires to manipulate particular receptors so as to best manipulate the human body. This process of mechanism manipulation is considered a pathway definition for which medications are classified, often noted by mechanism of action or by pharmacologic therapeutic class.

Some mechanisms share a closer similarity in altering different parts of the same pathway. Such mechanisms are less unique for the more practical purpose of this patent. Mechanisms distinguish various pharmaceutical composition attempts to treat disease. They are distinguished as defined unique mechanism classes.

Within a mechanism class, there can exist subclass distinctions. Such subclasses are lessened in uniqueness as they effect similar mechanistic pathways. If a mechanism is not unique enough to prescribe simultaneously with another similar mechanism with the current conventional model, treating a given patient for a given disease, then its use in this patent could require a clinical caveat. Such a less common caveat may be most notable with the renin-angiotensin system as noted in List 2b, but such a caveat of using a subsystem calculation is of seemingly minor practical importance.

New receptors are continually being discovered, as are novel ways to manipulate those receptors. Searching for the latest mechanism manipulation is very much the endeavor for which current research and development resources are geared. This limited receptor process, manipulated with potent doses, neglects the knowledge that too often many receptors are involved in a particular disease process. An overly predictive preoccupation with singular data points reflecting disease outcomes adequately, often represents highly intricate efficacy manipulation, and is highly associated with the wasteful aspect of modern medicine. The exorbitant hoodwinking involved in clinical trials, evaluating such potent medication reactions, is notable to mention for it is relevant to the advancement of cooperative medication, but beyond the scope of detailing the high level cooperative combination method requiring lesser initial doses and the rational process of maximizing benefit with better risk aversion.

As it stands, the current model of medically treating a given condition lacks integrity. One way this is well documented is in the 1999 Institute of Medicine research, To Err is Human. From the IOM report, the extensive and costly ramifications of medical errors were exposed with considerable detail. It is noted most medical errors occur at the level of prescribing pharmaceutical compositions, often involving dosing errors. Research and development resources have been heavily skewed toward composing a single pharmaceutical compound, the current model referenced herein, and the treatment options for the most studied, most profitable diseases have consequently become most excessive. This has been the status quo, seemingly accepted for decades, as the research and development for the approval of such a given compound is a massive undertaking, making such development feasible only for those wielding tremendous budgets. Data points in turn can appear favorable to those with high budgets. After further brief oversimplification explanations, it may become clearer why the novel cooperative model, a pharmaceutical composition of calculated proportional doses, is revolutionary and is rational to exponentially shift resources toward such an improved system of development. This cooperative medication combination system rationally presupposes already accepted data points.

Combining potent medication therapy into a composition for a particular indication is not new. Mere potent medication combination is more of an obvious step in an evolving pattern of treating a given disease requiring multiple individual medications, especially where patentability of such profitable compositions is involved. The novelty of the cooperative combination medicine system, a pharmaceutical composition of calculated substandard proportional mechanisms, reinvents the entire research and development process, current potent combinations included. In other words, the less than clinically significant distinctions among pharmaceutical compositions that are commercially available or otherwise allowable by various regulatory agencies are not well paralleled with this advanced method of disease based treatment.

Similarly, the combination of medication, creating a composition to best mimic desired human physiology is not new. Oral contraceptives are an example of combinations of medications sought to best mimic the balance of the human body, albeit in a way for which body physiology is tricked. A basic contraceptive combines an estrogen equivalent with a progesterone equivalent. The numbers of combinations using some combination of just one type of estrogen, namely estradiol, are greater than 100. The number of products available in the area of contraception illustrates a great deal of the current research and development model's lack of economic sustainability. The various contraceptive products differ more in slight nuances of little clinical importance; they are less of a pharmacopeia of various chemical mechanisms to prevent pregnancy than they are a collection of different chemical structures, all of which are designed merely to target the very same receptors to prevent ovulation. The different chemical structures marketed tend to collectively act upon the very same receptors. Oral contraceptives are a good example of how the current conventional medication model, including combination therapy, encourages resource ineffectiveness, regardless of its efficacy in preventing ovulation. In fact, the ISMP errors created by such patterned instances of abundant options likely outweigh the less than clinically significant variety of options available.

As is the case with hormonal therapy, the exceptionally intricate chemical balance of the human body is likely not nearly as well understood as marketing current research would suggest. Data points are manipulated. If one company develops a rudimentary mechanism model, then the competitor generally aspires to do the same, and does so rapidly, so as to not lose market share. If the new cooperative model of creating a pharmaceutical composition, contained herein, was implemented, then that entire process would fluctuate. Most notably, the conventional single chemical model would fluctuate in the direction of what is significantly greater for the patient, most notably treating an indicated illness, and profit would be a more of a secondary effect.

Glaring examples of modern medicine blinded by convention, via marketing, data point abuse (absolute data versus relative data value illustrative distortions, emphasis of less than primary study endpoints, lack of statistical power, distraction from side-effect profile concerns, warped method standards, lack of competitive head to head trial data, etc.) is reflected in very simple, less profit oriented discoveries. It was not until after 2007 that paracetamol doses were conventionally recommended to be reduced for patients with kidney dysfunction. That took roughly 130 years after discovery of one of the most widely used and widely essential, per the World Health Organization, medications in modern medicine for such a significant dosing adjustment to be declared.

One of the most successfully marketed and widely used medications was not handled with appropriate scrutiny for peculiar reasons. The same medication, used in combination, where the only difference compared to another multi-medication formulation was aspirin exchanged for acetaminophen (paracetamol), while both multi-medication combinations were granted legend status, the aspirin combination is still additionally labeled as a DEA (Drug Enforcement Agency) controlled substance to this very date. The paracetamol combination is not labeled a controlled substance. In so doing, aspirin, an over the counter product, is essentially labelled a DEA controlled substance via this combination regulatory status, as it is the only difference in the butalbital compound that is not legally a controlled substance when aspirin is simply replaced with acetaminophen in the very same surrounding ingredients. Improvements in the advancement of medicine ingenuity requires more critical thinking than statistical data devoid of higher order reasoning.

A fixed dose combination tablet, generally referred to as a polypill, used in the UMPIRE trial, aspires some degree of novelty as it sought to achieve better outcomes than its individually prescribed chemical composition. Like the TIPS trials, the polypill combined doses of various mechanisms used to treat cardiovascular disease, included was a 3 mechanism regimen of hypertension medications, with a cholesterol medication and an over-the-counter (OTC) clot prevention medication. It is understood that such a combination is for convenience, increasing the medication regimen adherence of the patient, lending itself to greater efficacy with the seemingly consolidated cocktail used for medication administration compliance. Although it is somewhat of a novel idea to combine aspirin and a statin with a few hypertension medication staples, it is much different than using substandard doses to attempt a synergistic effect of consistently lower blood pressure, while decreasing given side-effects. The combination of various medications is not new. Combinations are most often done to evade patent expirations, under the guise of ease of patient administration compliance.

The TIPS-2 trial added potassium to its polypill, an oral pharmaceutical for which hypertension is treated with potent doses of antihypertensives. This addition of potassium has been an addition that could make sense in most potassium depleting diuretic regimens. The addition of potassium would seem to make sense to any clinician familiar with diuretic treatment of hypertension, but it is just not cost-effective to research and develop a new antihypertensive medication that simply adds potassium. Diuretic tablets are generally the cheapest medications to use orally for high blood pressure, and the clinical inertia to use a costly new, albeit more novel potassium containing diuretic would generally not outweigh the cost to use older diuretics that do not contain the potassium replacement.

The novelty of the polypill is subtle, but it takes advantage of the more recently noted compound diseases that compose general cardiovascular disease, and instead of using the different tablets for the different standard diseases encompassing cardiovascular disease, the polypill offers one tablet that would seem to fit well with most patients suffering from general cardiovascular disease. The hypertension aspect of cardiovascular disease offered a three mechanism approach to lowering blood pressure. So far, the polypill is the best attempt to advance the soon to be archaic potent single active medication archetype.

The polypill is composed of higher doses than general practitioners typically use for newly diagnosed hypertension. The polypill treatment would be more convenient for a given cardiologist seeking to treat a given cardiology patient on established therapy. As healthcare is further fractionated into specialties, specialists such as cardiologists tend to see general health care through a narrower lens, albeit a highly intricate specialized lens. It would make sense that the more lucrative aspects of healthcare, such as cardiology resources, have progressed the most (See List 1a and 1b for extensive pharmaceutical treatment options available). However, with all the resources available, it would be prudent to begin to shift to the more efficient wide scale use of the cooperative medication system method (See Table 1 “Syncpress” pharmaceutical tablet created with algorithm based substandard doses to treat hypertension).

The new cooperative combination model assumes human chemistry is a form of chemical balances. The chemical forces are seen more in light of the extensive chain of events resulting from alterations in the basal human chemical milieu. General concepts of diurnal patterns of chemical activity, such as the role melatonin, growth hormone, and the adrenal gland's adrenaline and cortisol, can each be analyzed to express a lifetime of minutia and subtle intricacies that could still leave many significant questions unanswered. The diurnal human chemical pattern is just one generally simple chemical process. There are seeming countless others, especially when pathologic pathways are examined, and the quantity is exponentially greater when juxtaposed with medicinal influences. The wealth of sensory apparatus, affecting human chemical balance, such as the common experience of mere auditory music, visual aesthetics, or olfactory stimulation bestow alterations in the chemical activity of a human body. Such further abstract seeming phenomena, sensory apparatus experience, experience not generally considered ingested, scratches at the human chemical grandeur beyond the scope of either model, but a looming confounding reality nonetheless, regardless of the method treating illness.

Any given bodily complexity, such as the totality of adrenaline's effects throughout the body, especially at the various cellular levels, would seem to be understood to a lesser degree than the status quo presumes. Perhaps the not so archaic words of Thomas Edison still hold some truth: Until man duplicates a blade of grass, nature can laugh at his so-called scientific knowledge. Further simplifications of the human body will still help to reveal the underlying complexity and how to best follow advances in understanding. The direct and compensatory mechanisms and such are thought to follow Newtonian laws, in ways which are not always completely understood, to further a chain of chemical events in multiple ways throughout the human body. For example, a given chemical manipulation of cell receptors at a given location of the human body accompanies a series of events. Some of these chemical events are understood more thoroughly than others. This includes the psychological manipulation of the human mind to alter the intricate chemical balance of the physical human body and vice versa. Nevertheless, the cooperative model is more advanced and more physiologically representative than previous disparate conventional corporate interpretations, taking into account more of that which is readily therapeutically available today. Until now, no method to adequately capture current technology has been feasibly detailed.

Generally, the current model of disease treatment with pharmaceuticals uses potent chemical mechanism manipulation. The current medical model is associated with an increased life expectancy in areas infiltrated with the methods of modern medicine. The trend of chronic medicine use has a noted lessened efficacy after decades of treatment for a given individual. It is rational to see this related to the sheer economic incentive to make medications potent to rapidly show evidence of effects via simple data points. Efficacy rates over extended periods are more of a quality of life issue than the concern of modern corporate medicine processes, as corporate and regulatory agendas do not currently adequately coincide with quantified methods of extended quality of life, especially insidious chronic disease. This pattern of lessened efficacy rate over extended periods is noted in various disease states, formulation of pharmaceutical, and/or particular potent mechanism being targeted with modern medicine.

To relate the difference between the current fractionated medical model, it is helpful to consider a given antihypertensive medication. Perhaps the most efficient is a simple diuretic such as hydrochlorothiazide. Diuretics generally promote urine production. Hydrochlorothiazide facilitates sodium loss from the plasma and extracellular fluid, via kidney receptors, that releases water and lessens the pressure on the vasculature, as measured with sphygmomanometry. There are various general phases of how diuretics affect blood pressure. There is an initial phase of blood volume loss, but the body learns to compensate for this loss. Eventually diuretics result in vasodilation from an unknown mechanism. Through these mechanisms various hormones are affected. Included in the diuretic effects are alterations in uric acid, glucose, potassium, calcium, sodium, chloride, bicarbonate, hydrogen, etc. The consequent effects of altering these various substances associates diuretic use with gouty arthritis, diabetes mellitus, hypokalemia, osteoporosis, dehydration, neurologic disorders, metabolic pH changes, hypotension, etc. Generally, diuretics are considered a simple and distinct mechanism (albeit there are various subtypes of diuretics), altering sodium mechanics in the kidney, and they are deemed clinically safe for the general population. The simple distinct method of increasing urine production does influence various systems in the body; of primary note is the antihypertensive effect reducing blood pressure. The other areas affected are not so well studied.

It is important to note that double/triple-blinded placebo controlled clinical studies do not adequately capture efficacy rates over extensive periods of time, periods of medicinal use lasting decades. Epidemiological studies come closer to expose extensive periods of time and the use of potent pharmaceutical compositions to treat disease over that extended time period. Periods of extended time are lacking in the evaluation process in regards to standard dose indication approval through regulatory agencies. A further look into the financial reasoning aspect of this lack of understanding may be helpful, but is not the intent of this patent.

As a government agency regulating the efficacy and safety of prescription medications, the Food and Drug Administration (FDA) requires the entity applying for approval of a given medication to provide data regarding minimally effective treatment, or starting doses for an indicated malady for which the medication has a demonstrative effect. The FDA has standards that can be based on a surrogate endpoint for more conveniently quantifiable results. An allowable prescription medication has standards of dosing and indication based upon such isolated convenient endpoints. These standards are significant for this patent. For hypertension, blood pressure readings can be established and the medication's resulting reduction compared to placebo is measured and the dose dependent phenomena allows for a minimal dose to be declared effective, sometimes a maximum is determined. This can likewise be done for blood sugar, cholesterol, and standard quality of life surveys, gastric acid, histamine release, brain waves, sleep patterns, ocular pressure, continence issues, pain scales, standard laboratory tests, movement patterns, tender point palpation analysis, imaging technology, electrographic technology, etc, for a given malady studied.

A notable dose of a “low” dose is often referenced in medicine. This invention refers to standard low doses and standard indications. For the purpose of this method of invention, the standard low dose is the lowest dose on a New Drug Application (NDA) at the FDA, and it would be the lowest dose manufactured as a prescription. Likewise, a standard indication comes from a NDA and would be the indication manufactured for prescriptive use. Off label use of a prescription may be included in the cooperative combination medication system, but its inclusion is via claim 2 of this invention since such use is not standardized to the specification of claim 1, claim 1 involving standard doses for standard labelled serious indications.

Serious disease often has a labeled prescription pharmaceutical composition for which the disease can be treated by those licensed to treat such serious disease. Serious treatment of serious disease has standards. Standard doses and indications are a function of the FDA. Only those with extensive education in the art of medicine, verified with certification from regulatory agencies, can prescribe within the standards of the art of medicine. This distinction relates to why an OTC NDA is not part of claim 1 of this method of combining substandard doses to create a pharmaceutical composition that treats disease.

A standard dose has a standard indication and standards of pharmaceutical composition. These are related in a practical sense via algorithm and FDA prescription patent standards. The algorithm is a calculated proportion via this method of invention. The standards of both an indication and dose are related as both required distinctions are included in the invention method. Both indication and dose are both standards already defined by FDA NDA standards.

It may be true that patent and FDA standards allow for less than clinically significant drugs or pharmaceutical compositions to gain approval, but the standards of dose and indication are the best available, and are therefore the standards of practice and theory. Such doses and indications are the guides in tertiary literature. A substandard dose is a dose based upon both a standard dose and a standard indication, standards found for legend/prescription medications in readily available professional tertiary literature. The standard distinction is crucial for distinguishing claims 1 and 2 of this application. The FDA makes the distinction clear with the prescription process, so a cooperative medication combination is clear via the combination method of claim 1, and such a combination can be further improved with claim 2.

Some prescription medications leave the legend status. In other words, less frequent exceptions can allow a prescription medication to become over-the-counter (OTC) via a different FDA application. The low dose of such an instance, for the purpose of this invention, would still be the lowest dose of the FDA NDA, but such a medication could theoretically qualify for inclusion of a substandard dosed portion (i.e. part of claim 1 of this application) of the cooperative combination herein if dosed within the substandard parameters, parameters outlined in a further description of substandard below. Otherwise, such a rare case of a prescription becoming OTC could be included as a generally detailed less standardized OTC portion of the cooperative combination composition (the lesser standardization is the position of OTC by definition not monitored by medical professionals to standards of medical knowledge for serious disease, disease requiring a prescription to obtain behind the counter medication) via claim 2. Such a component of a pharmaceutical preparation could theoretically meet both the criteria of having a substandard dose and likewise be OTC. Albeit no such practical entity is known to exist, and is not likely to occur for reasons already addressed. For the purpose of defining this method of invention: a prescription medication that is later approved for OTC use, and is proportionally dosed at a standard lesser than its low dose on the original NDA, and it is combined in a pharmaceutical composition with at least 2 other such substandard dosed medications, it would meet the inclusion for the method of invention, an invention described further below.

Of a similar note, off label prescription medication use is when a legend/prescription medication is used for an indication for which it was not approved, a legal occurrence allowable within the artistic scope of a legal prescriber. In this instance, the off label use of prescriptions would not count as one of the minimal standardized 3 medications for claim 1. If, in the unlikely event the off label sought formal approval for the previously off label indication, then it could then be used as a component of claim 1.

The calculated proportion of the cooperative medication combination system refers to the division of a series of low doses, the series quantified by the number of unique chemical mechanisms to be combined with substandard doses. This method minimally requires 3 such medications of unique mechanism (mechanisms less distinct, involving similar pathways, could be further fractionated to equate an effect only as great as its proportion related to the total number of unique mechanisms), so a standard low dose would simply be divided by 3 for such a composition of 3 medications dosed at roughly ⅓ of its given standard low dose. Therefore, the proportional calculation of a series of substandard doses would make for a combined composition potent enough to treat the totality of complex diseases, but does so in a more complete fashion. The cooperatively combined substandard doses would be the improvement of treating the same standard indication individually treated by the standard individual low doses.

For instance, if an extended release pharmaceutical composition of 2.5 mg of omeprazole, 2.5 mg of famotidine, and 250 mg of sucralfate to be ingested twice daily made clinical sense for treating gastrointestinal erosion, then even though omeprazole and famotidine are currently OTC, they were previously standardized to have a low dose via NDA prescription vetting, and would be included as a composition defined by the method of cooperative combination systems. Likewise, if loratadine 2.5 mg, montelukast 2.5 mg and prednisone 0.25 mg were combined in a pharmaceutical composition to ingest orally for allergy symptom treatment, then that substandardly dosed composition would too meet the minimal definition, even though loratadine is now available off the legend status. If either the gastrointestinal or allergy treatment pharmaceutical compositions above used the now OTC products in standard OTC doses but added substandardly dosed medications of differing mechanism in the place of the now OTC medications, a composition still containing at least 3 substandard dosed medication exists, then that new composition too would meet the requirements for the method composing a cooperative medication system.

For the teaching aspect of this patent, a pharmaceutical composition can be referred to as medicine. A pharmaceutical composition can vary in the dosage form. Oral formulations are most common, so this patent provides more oral formulation teaching examples. Otic, ophthalmic, nasal, injectable, rectal, and topical applications are likewise pharmaceutical dosage forms, but such medicinal instances of use are less as there are far fewer options than there are orally ingested mechanisms. Oral pharmaceutical compositions may consist of tablets, capsules, caplets, perles, sustained released (noted as timed release, delayed release, controlled release, biphasic release, coated, bead release, extended release, long-acting, XR, XL, ER, TM, etc.), immediate release (orally disintegrating, effervescent, sublingual, wafer, etc.), lozenges (buccal, disc, sucker, etc), solution, suspension, emulsion, powder, etc. Any alleged clinical distinction of such dosage forms is not the intent of this method of invention, but the aim of this paragraph is to simply note various pharmaceutical compositions, reference the similarity to medicine, and to emphasize oral forms are most common.

However, such a cooperative combination of ophthalmic preparations may well be a not only feasible treatment option, but would likely be the best option for glaucoma patients well on the way to losing one's entire visual field. One of the most reliable ophthalmic pharmaceutical glaucoma treatment staples has been used as a staple for most glaucoma patients for more than a decade. When it becomes less effective, other older additions can preserve some years of vision for qualified patients. After that period of attempted salvaging, many patients have next to no option other than living in legal to complete blindness. Using the method of cooperative combination therapy, one could use the algorithm to calculate a proportion likely even less than ⅕ for 5 of the most feasibly distinct ophthalmic mechanisms available to treat glaucoma. The ophthalmic composition can consist of a prostaglandin analog, a beta blocker, a cholinergic, an alpha-2 adrenergic stimulator, and a carbonic anhydrase inhibitor. The combination of these formulations, in proportionally less doses of unique mechanisms to create a further pharmaceutical composition, can quite literally save thousands from eventual blindness. It is likely to even prevent the worrisome systemic effects for which some potently dosed mechanisms are associated, and their consequent conventional high dose use is therefore avoided. A final addition of acetylcysteine can further improve the cooperative medication combination system treatment of glaucoma.

Likewise, hypertensive emergencies can best respond to a cooperative combination of substandard injectable antihypertensives, yet another non-oral composition. A composition consisting of a series of substandard dosed medications may allow for more aggressive treatment, saving eye damage, kidney damage, stroke, cardiac damage, and prevent death. The calculated proportion of ¼ of a low standard indicated dose of a beta-blocker+alpha blocker, an angiotensin-converting enzyme inhibitor, a centrally-acting adrenergic agent, and a vasodilator makes a feasible pharmaceutical composition treating high blood pressure. Both the ophthalmic and injectable pharmaceutical compositions above use readily available prescriptions to further compose a complete disease state composition.

Standard indicated starting prescription doses are well documented in readily available tertiary literature, manufacturer package inserts, etc. This application does not list the thousands of pharmacopoeia standard options available, since this is a method of utility being emphasized. The standard indicated starting doses are referred to as being clinically effective, and too are readily available by reference. It is not the standard of current practice to begin treatment with multiple mechanisms as this invention details.

Doses below the designated effective dose, below a low dose, are understood to not be clinically effective in isolation, and are consequently not used as novel effective treatment options, certainly not in combination. These substandard doses generally do have measurable effects, subclinical effects, effects just not to the degree sought for standard indicated thresholds. Generally, substandard doses, as referred in this patent, are those that are lower than the lowest manufactured or approved dose. A more numerically concise range of a substandard dose would be at least 5% less than a standard indicated low dose (95% or less than 95% of the standard low dose), but also 5% of the standard low dose greater than a 0 quantity (5% or above 5% of the standard low dose), making the substandard dose quantity 5-95% of a low standard dose intended for a 24 hour period. The substandard range is distinctly defined as the 90% quantity directly between a standard low dose and zero. Such a 90% range defined as substandard makes for a relative maximum of 20 distinct or unique mechanisms to combine substandardly in the algorithm. If 21 unique mechanisms were at all feasible, one could still theoretically make the resulting substandard doses, each 5% or greater to still remain within the substandard limits. The minimum of 3 mechanisms in a cooperative medication combination system is an absolute.

Substandard herein is determined via an algorithm equal to the process of fractionating a low standard dose, a low dose indicated to treat the same disease process as the series of components to likewise be fractionated and henceforth combined into the final pharmaceutical composition. The calculated algorithm determining a substandard dose takes the low dose and divides it by the total number of cooperative medications to be combined into the final composition of a series of unique mechanisms of action. All mechanisms of action will be treating the same disease. The parameters of the substandard doses are expressed above with the 5% designations, leaving a 90% range for a substandard dose to be between a low dose and a zero dose. This algorithm is a critical step in calculating the proportion for a method to manufacture pharmaceutical compositions that offer improved utility in disease treatment.

Given that doses lower than an FDA designated low dose are not alone deemed clinically effective, they are not typically manufactured as their use is not a standard of practice. Homeopathy may appear to use substandard doses; however, it does so to such an extreme that the “active” chemical has been subject to such monumental dilution that it has subsequently lost all rational efficacy, outside of psychogenic placebo effects. These homeopathic doses are significantly further below 5% of a standard indicated low dose, and are much closer if not equal to a 0 quantity. The new method calculates doses wholly distinct from both homeopathy, and also “low” combination doses that are noted in some current conventions of therapy. Substandard doses in the cooperative combination would fall between these two dose ranges, the range of homeopathy and already delineated “low” doses, whereby the dose of a single substandard dose has a measurable effect, just not one that meets the FDA cutoff for clinical efficacy when used in isolation (i.e., without additional ingredients used cooperatively to meet a clinical goal). The calculation of such a substandard dose would generally be as high as 33% of a low standard indicated dose, but again would fall within the boundary of 5% above a 0 quantity and 5% less than a low standard indicated dose. The model that even minimum low dose prescriptions offer a single chemical potency that significantly alters a fraction of a “known” chemical/mechanical physiologic pathway to such a high degree that the results are deemed clinically significant by the FDA is more associated with the substandard distinction herein.

A clinical goal achieves a given indication while lessening clinically significant side-effects, and does so to the greatest degree of available alternatives. The cooperative combination of proportionally calculated substandard doses advocates not just clinical significance, but best achieves a goal with an advanced method for treating disease with a pharmaceutical composition. In the case of the method of cooperative combination therapy, the benefits of such resulting composition is much further reaching than is typically required or even evaluated for an FDA NDA. This new method aims to not only improve safety and efficacy, but does so with direct pharmacologic mechanism and related prescriptive safety, efficacy rate improval over extended periods (decades), and conserves resources.

When a series of substandard dosed cooperative mechanisms are used to reach one endpoint, such as lower blood pressure (preferably lessens mean arterial pressure), then the various mechanical distinctions are likely rational to have a synergistically positive effect. In other words, the compensatory mechanisms and time dependent associations with a given chemical can result in a smoother omni-therapeutic result when combined with that which has already been proven to be effective individually.

The inverse would be true for a given side-effect particular for a given mechanism, whereby the substandard combination would result in a diminished incidence of any particular side-effects for a given unique pharmacologic mechanism. With a lower dose used to accomplish a collaborative endpoint, the resultant individual dose dependent side-effect is proportionally less. In the case of a diuretic, the metabolic elimination can further effectively reduce the potentially troubling elevations in uric acid, glucose, etc. otherwise found with higher potent diuretic concentrations conventionally utilized.

It is rational that common side-effects among mechanisms will additionally be lessened as the cause of the common side-effect is occurring at a much different rate, time, and magnitude than when occurring with a single mechanism model utilizing higher single-drug potency, as the high potency single medication has an intense uniform, albeit only partialized physiologic impact. Hypotension is a common side-effect among antihypertensives. This side-effect would occur with a lesser velocity and consequently to a lesser adverse degree when six mechanisms are only synchronized to lower the blood pressure, not to do it in a collectively organized intense kinetic time frame, beyond the time frame for which it is already studied and approved. Similarly, there would be a mitigation of rebound hypertension if tapering off the cooperative medication system was desired. This dispersive but collective effect too would be the case with a cooperative combination system used to lower blood sugar or symptomatic alleviation with a depression treatment model of this new design.

The novel art described herein with active steps is empirically rational. It has the potential to radically change the way medical disease is managed, and in a convenient cost-effective manner. As a rational process, it is described in lengthy terms, using already available technology. The ramifications, both therapeutic and also economic, are difficult to fully quantify. It is anticipated that the invented pharmaceutical compositions will help to further illustrate this novel method with concrete examples and further explanation via concise active steps. This paradigm shift can reasonably take much of the practice out of medicine, for it utilizes medications that are already understood to a significant degree, but uses them more effectively, safer, earlier in the disease process, and in a way for which the efficacy has greater longevity. The medications are even well understood when used together, albeit currently only at individually therapeutic or even a low potent dose, often when treatment with a given single unique mechanism has proven inadequate. The cooperative method is reasonable to provide unexpected results because it is of greater rational consequence than the current conventional method, and consequently would better address that which is poorly measured with the current method of pharmaceutical composition use. In some ways this new cooperative method for therapeutic treatment is a holistic form of conventional medical therapy, an unequalled fusion of that which is already clinically available, but yet to be concisely detailed. For the sake of pedagogy, practical examples are henceforth provided below.

### BRIEF SUMMARY OF THE INVENTION

The invention method is that of combining pharmaceutical compositions, treating patients with beyond lower than manufacturer/government entity determined “low dose” medications, and does so with an algorithm calculating a cooperative combination of no less than three combined substandard dosed pharmaceutical compositions. The reason for combining medications is to achieve more clinically significant results from a variety of substandard proportioned doses of medications with unique mechanisms of action. This combination may include the addition of vitamins, minerals, supplements, non-legend medications (OTC), off label prescriptions, or nutraceuticals. When used individually, the proportionally lower dose of the medication would merely be generally considered clinically insignificant, for the general population, at such a substandard dose. By combining multiple medications into one pharmaceutical composition, each in their own substandard dosage, it is rational that the resulting cumulative combination composition would produce markedly clinically significant efficacy, all while precluding side-effects and interactions for certain indications, the collaboration of which reinvents pharmacological prescribing practices.

### BRIEF DESCRIPTION OF THE SEVERAL VIEWS OF THE CONCEPT

FIG. 1 graphically quantifies and solidifies the rational nature of teaching the details of the cooperative combination medication system utility. Any of the medication prototypes created from Table 1-4 (not to exclude management of chronic pain, osteoarthritis, neuropathic pain, seizure disorders, constipation, insomnia, hypertensive emergency/urgency, gastrointestinal erosion, glaucoma, allergy symptoms, fibromyalgia, schizophrenia, Parkinson's, dementia, emesis and nausea, etc. of this genus type method of utility) could be applied to the figure, with the conventional low dose medication corresponding to A, B, C, etc. Likewise, the algorithm derived substandard component doses of the cooperative combination medication systems correspond to A (ss), B (ss), C (ss), etc, and are noted to have sub-clinical responses individually. The figure demonstrates the combination of all of the substandard components would produce an improved indicated effect, as seen with the last graph bar labeled “Cooperative Prototype.” This greater indicated effect would therefore be greater than the standard indicated effect. This represents the clinically rational measure of cooperative combination medication systems compositions. The dotted line designated as “1” is the indicated effect of that of a low dose medication, a designation of clinical efficacy. This represents the general dose manufactured by a given industrial entity, the lowest dose on an NDA application. The dotted line designated as “2” represents the minimum threshold for which the Federal Food and Drug Administration approves a clinically significant indicated effect. Line “3” demonstrates the anticipated indicated effects measured by homeopathic doses of medications, doses less than 5% of a low dose.

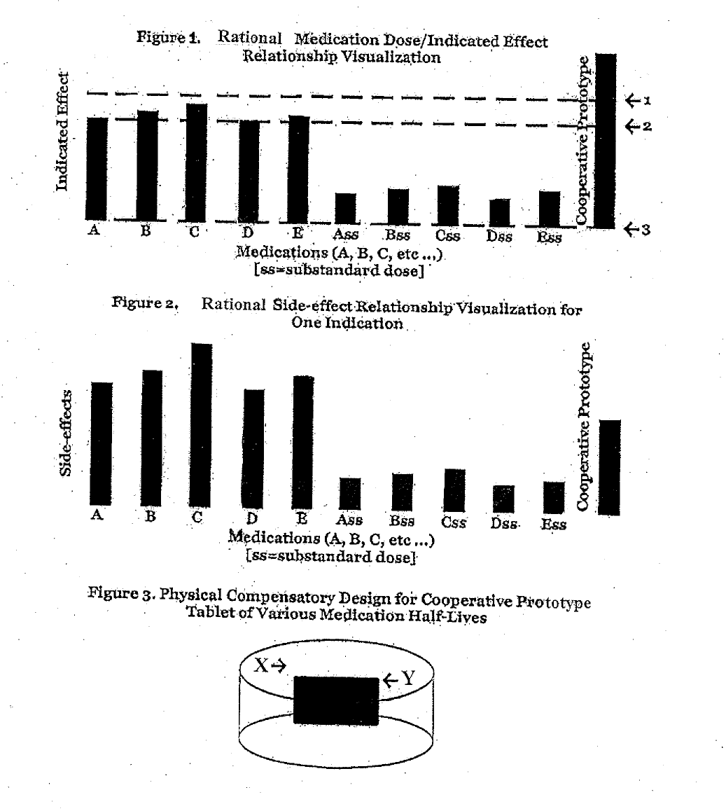

FIG. 2 is similar to FIG. 1, except FIG. 2 compares the same algorithm delineated dosages in relation to side-effects. This time the medicinal kinetics illustrate a dynamic that renders the cooperative combination medication system prototype composition with a comparatively lessened side-effect profile. Likewise, this graphically represents the ramifications of the composition, with many moving clinical parts.

FIG. 3 describes a medication delivery system dosage form that further illustrates the pharmaceutical composition of the invention. “X” is a tablet exterior containing the immediate release long half-life medications, including ¼ of the total dose of short-acting medications, vitamins, minerals, supplements, etc. “Y” is a sustained delivery matrix (i.e. potential methylcellulose composite) releasing ¾ of the total short-acting medications over an extended period of time, providing a sustained 24 hour effect.

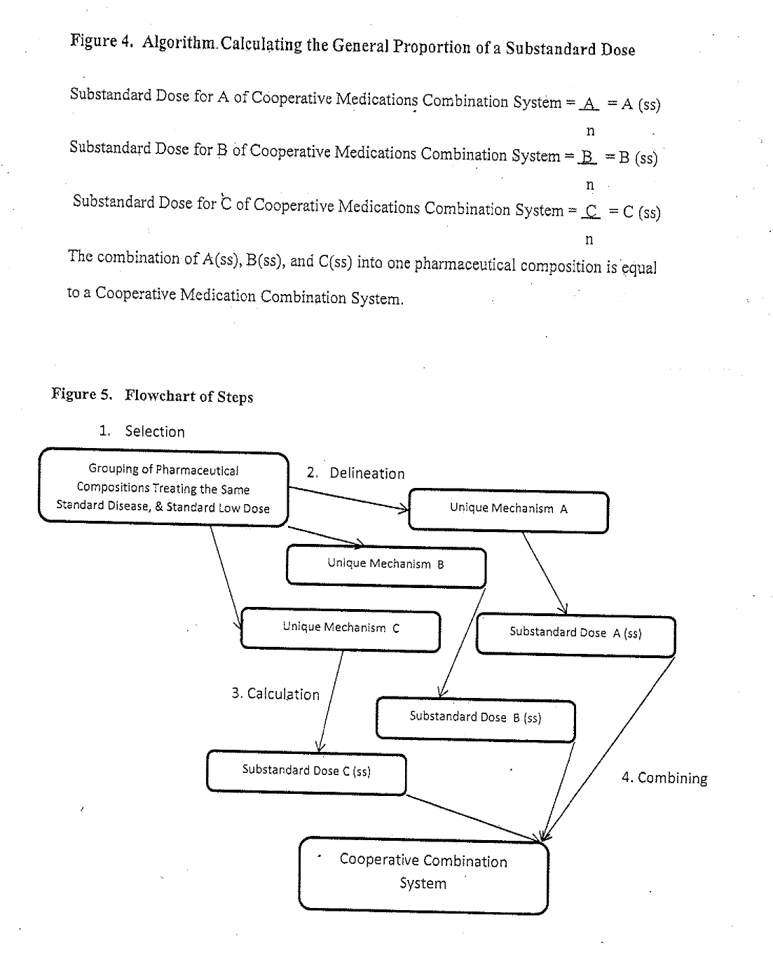

FIG. 4 is the algorithm of calculating the general proportion of a substandard dose. A, B, C, etc, are low doses of various unique mechanisms of pharmaceutical compositions treating the same disease. A (ss), B (ss), C (ss), etc, are substandard doses of the various medications to now be combined into one pharmaceutical composition to treat the same disease. The “n” is equal to the number of unique mechanisms in the cooperative medications combination system. A mechanism of action is taken from standard therapeutic classification, many are copied in Lists 1a, 2a, 3a, 4a . . . . This example of a cooperative medication combination system contains 3 unique mechanisms of action, so n=3. Therefore, the substandard doses of A (ss), B (ss), and C (ss) would be roughly 33% of a low dose.

FIG. 5 is a flowchart of the steps involved in the method creating a cooperative combination medication system.

TABLE 1
“Syncpress” - teaching example for composition calculation
for hypertension treatment

| Medication | Substandard | | 5 times | | “Low” | | | |
|---|---|---|---|---|---|---|---|---|
| name | Strength (SS) | | SS | | dose/24 hrs | | Half-life | |
| Carvedilol | 1 | mg | 5 | mg | 6.25 | mg | 9 & 7 | h |
| Lisinopril | 2 | mg | 10 | mg | 2.5 | mg | 12 | h |
| Chlorthalidone | 4 | mg | 20 | mg | 25 | mg | 50 | h |
| Amlodipine | 0.8 | mg | 4 | mg | 2.5 | mg | 40 | h |
| Clonidine | 0.02 | mg | 0.1 | mg | 0.2 | mg | 12 | h |
| Hydralazine | 5 | mg | 25 | mg | 40 | mg | 5 | h |

TABLE 2
“Mellyces” - teaching example for composition calculation
for diabetes mellitus treatment

| Medication | Substandard | | 5 times | | “Low” | | | |
|---|---|---|---|---|---|---|---|---|
| name | Strength (SS) | | SS | | dose/24 hrs | | Half-life | |
| Metformin | 200 | mg | 1000 | mg | 1000 | mg | 6 & 17 | h |
| Sitagliptin | 12.5 | mg | 62.5 | mg | 25 | mg | 12 | h |
| Pioglitazone | 4 | mg | 20 | mg | 15 | mg | 5 & 20 | h |
| Glyburide | 0.3 | mg | 1.5 | mg | 2.5 | mg | 10 | h |

TABLE 3
“Eclepid” - teaching example for composition calculation
for dyslipidemia treatment

| Medication | Substandard | | 5 times | | “Low” | | | |
|---|---|---|---|---|---|---|---|---|
| name | Strength (SS) | | SS | | dose/24 hrs | | Half-life | |
| Simvastatin | 4 | mg | 20 | mg | 5 | mg | 2 | h |
| Fenofibrate | 5 | mg | 25 | mg | 54 | mg | 20 | h |
| Ezetimibe | 0.5 | mg | 2.5 | mg | 10 | mg | 22 | h |

TABLE 4
“Lucidotion” - teaching example for composition calculation
for symptom of depression treatment

| Medication | Substandard | | 5 times | | “Low” | | | |
|---|---|---|---|---|---|---|---|---|
| name | Strength (SS) | | SS | | dose/24 hrs | | Half-life | |
| Sertraline | 4 | mg | 20 | mg | 25 | mg | 24 | h |
| Venlafaxine | 15 | mg | 75 | mg | 75 | mg | 5 | h |
| Amitriptyline | 4 | mg | 20 | mg | 25 | mg | ~53 | h |
| Mirtazapine | 2 | mg | 10 | mg | 15 | mg | 30 | h |
| Trazodone | 4 | mg | 20 | mg | 25 | mg | 4 & 7 | h |
| Methylphenidate | 0.25 | mg | 1.25 | mg | 2.5 | mg | 3.5 | h |
| Aripiprazole | 0.25 | mg | 1.25 | mg | 2 | mg | 75 | h |

List 1a. Available Prescription Hypertension Medications with Mechanism Class:
Acebutolol—Beta-Blocker (BB)
Aliskiren—Direct Renin Inhibitor
Aliskiren; Amlodipine—Combination of Direct Renin Inhibitor/Calcium-Channel Blocker (CCB)
Aliskiren; Amlodinine; Hyvdrochlorothiazide, HCTZ—Combination of Direct Renin Inhibitor/Calcium-Channel Blocker (CCB)/Thiazide Diuretic
Aliskiren; Hydrochlorothiazide, HCTZ—Combination of Direct Renin Inhibitor/Thiazide Diuretic
Aliskiren; Valsartan—Combination of Calcium-Channel Blocker (CCB)/Angiotensin II Receptor Antagonist
Amiloride—Potassium-Sparing Diuretic
Amiloride; Hydrochlorothiazide, HCTZ—Potassium-Sparing Diuretic/Thiazide Diuretic
Amlodipine—Calcium-Channel Blocker (CCB)
Amlodipine; Atorvastatin—Combination of Calcium-Channel Blocker (CCB)/Statin
Amlodipine; Benazepril—Combination of Calcium-Channel Blocker (CCB)/Angiotensin-Converting Enzyme Inhibitor (ACE-I)
Amlodipine; Hydrochlorothiazide, HCTZ; Olmesartan—Calcium-Channel Blocker (CCB)/Thiazide Diuretic/Angiotensin II Receptor Antagonist (ANG2)
Amlodipine; Hydrochlorothiazide, HCTZ; Valsartan—Combination of Calcium-Channel Blocker (CCB) Thiazide Diuretic/Angiotensin II Receptor Antagonist (ANG2)
Amlodipine; Olmesartan Combination of Calcium-Channel Blocker (CCB)/Angiotensin II Receptor Antagonist (ANG2)
Amlodinine; Telmisartan—Combination of Calcium-Channel Blocker (CCB)/Angiotensin II Receptor Antagonist (ANG2)
Amlodinine; Valsartan—Combination of Calcium-Channel Blocker (CCB)/Angiotensin II Receptor Antagonist (ANG2)
Atenolol—Beta-Blocker (BB)
Atenolol; Chlorthalidone—Combination of Beta-Blocker (BB)/Thiazide Diuretic
Azilsartan—Angiotensin II Receptor Antagonist (ANG2)
Benazeoril—Angiotensin-Converting Enzyme Inhibitor (ACE-I)
Benazepril; Hydrochlorothiazide, HCTZ—Combination of Angiotensin II Receptor Antagonist (ANG2)/Thiazide Diuretic
Bendroflumethiazide—Thiazide Diuretic
Bendroflumethiazide; Nadolol—Combination of Thiazide Diuretic/Beta-Blocker (BB)
Betaxolol—Beta-Blocker (BB)
Bisoprolol—Beta-Blocker (BB)
Bisoprolol; Hydrochlorothiazide, HCTZ—Combination of Beta-Blocker (BB)/Thiazide Diuretic
Bumetanide—Loop Diuretic
Candesartan—Angiotensin U Receptor Antagonist (ANG2)
Candesartan; Hydrochlorothiazide, HCTZ—Combination of Angiotensin II Receptor Antagonist (ANG2)/Thiazide Diuretic
Cantopril—Angiotensin-Converting Enzyme Inhibitor (ACE-I)
Captopril; Hydrochlorothiazide, HCTZ—Combination of Angiotensin-Converting Enzyme Inhibitor (ACE-I)/Thiazide Diuretic
Carteolol—Beta-Blocker (BB)
Carvedilol—Beta-Blocker (BB)
Chlorothiazide—Thiazide Diuretic
Chlorothalidone—Thiazide Diuretic
Chlorthalidone; Clonidine—Combination of Thiazide Diuretic/Centrally-Acting Adrenergic Agent
Clevidipine—Calcium-Channel Blocker
Clonidine—Centrally-Acting Adrenergic Agent
Diltiazem—Calcium-Channel Blocker
Diltiazem; Enalapril—Combination of Calcium-Channel Blocker/Angiotensin-Converting Enzyme Inhibitor (ACE-I)
Doxazosin—Alpha-Blocker
Enalapril—Angiotensin-Converting Enzyme Inhibitor (ACE-I)
Enalapril; Felodipine—Combination of Angiotensin-Converting Enzyme Inhibitor (ACE-I) Calcium-Channel Blocker
Enalapril; Hydrochlorothiazide, HCTZ-Combination of Angiotensin-Converting Enzyme Inhibitor (ACE-I)/Thiazide Diuretic
Elerenone—Aldosterone Antagonist
Erosartan—Angiotensin II Receptor Antagonist (ANG2)
Erosartan; Hydrochlorothiazide, HCTZ—Combination of Angiotensin II Receptor Antagonist (ANG2)/Thiazide Diuretic
Ethacrynic Acid—Loop Diuretic
Felodipine—Calcium-Channel Blocker
Fosinopril—Angiotensin-Converting Enzyme Inhibitor (ACE-I)
Fosinopril; Hydrochlorothiazide, HCTZ—Combination of Angiotensin-Converting Enzyme Inhibitor (ACE-I)/Thiazide Diuretic
Furosemide—Loop Diuretic
Guanabenz—Centrally-Acting Adrenergic Agent
Guanthidine—Centrally-Acting Adrenergic Agent
Guanfacine—Centrally-Acting Adrenergic Agent
Hydralazine—Vasodilator
Hydralazine; Hydrochlorothiazide, HCTZ—Combination of Vasodilator/Thiazide Diuretic
Hydrochlorothiazide, HCTZ—Thiazide Diuretic
Hydrochlorothiazide, HCTZ; Irbesartan—Combination of Thiazide Diuretic/Angiotensin II Receptor Antagonists (ANG2)
Hydrochlorothiazide, HCTZ; Lisinopril—Combination of Thiazide Diuretic/Angiotensin-Converting Enzyme Inhibitor (ACE-I)
Hydrochlorothiazide, HCTZ; Losartan—Combination of Thiazide Diuretic/Angiotensin II Receptor Antagonists (ANG2)
Hydrochlorothiazide, HCTZ; Methyldopa—Combination of Thiazide Diuretic/Centrally-Acting Adrenergic Agent
Hydrochlorothiazide, HCTZ; Metoprolol—Combination of Thiazide Diuretic/Beta-Blocker (BB)
Hydrochlorothiazide, HCTZ; Moexipril—Combination of Thiazide Diuretic/Angiotensin-Converting Enzyme Inhibitor (ACE-I)
Hydrochlorothiazide, HCTZ; Olmesartan—Combination of Thiazide Diuretic/Angiotensin II Receptor Antagonists (ANG2)
Hydrochlorothiazide, HCTZ; Propranolol—Combination of Thiazide Diuretic/Beta-Blocker (BB)
Hydrochlorothiazide, HCTZ; Quinapril—Combination of Thiazide Diuretic/Angiotensin-Converting Enzyme Inhibitor (ACE-I)
Hydrochlorothiazide, HCTZ; Spironolactone—Combination of Thiazide Diuretic/Aldosterone Antagonist/Potassium-Sparing Diuretic
Hydrochlorothiazide, HCTZ; Telmisartan—Combination of Thiazide Diuretic/Angiotensin II Receptor Antagonists (ANG2)
Hydrochlorothiazide, HCTZ; Timolol—Combination of Thiazide Diuretic/Beta-Blocker (BB)
Hydrochlorothiazide, HCTZ; Triamterene—Combination of Thiazide Diuretic/Potassium-Sparing Diuretic
Hydrochlorothiazide, HCTZ; Valsartan—Combination of Thiazide Diuretic/Angiotensin II Receptor Antagonists (ANG2)
Hydroflumethiazide—Thiazide Diuretic
Indapamide—Thiazide Diuretic
Irbesartan—Angiotensin II Receptor Antagonists (ANG2)
Isradipine—Calcium-Channel Blocker
Labetalol—Beta-Blocker (BB)
Lisinopril—Angiotensin-Converting Enzyme Inhibitor (ACE-I)
Losartan—Angiotensin II Receptor Antagonists (ANG2)
Methylclothiazide—Thiazide Diuretic
Methyldopa—Centrally-Acting Adrenergic Agent
Metolazone—Thiazide Diuretic
Metoprolol—Beta-Blocker (BB)
Minoxidil—Vasodilator
Moexipril—Angiotensin-Converting Enzyme Inhibitor (ACE-I)
Nadolol—Beta-Blocker (BB)
Nebivolol—Beta-Blocker (BB)
Nicardipine—Calcium-Channel Blocker (CCB)
Nifedipine—Calcium-Channel Blocker (CCB)
Nisoldipine—Calcium-Channel Blocker (CCB)
Olmesartan—Angiotensin II Receptor Antagonists (ANG2)
Penbutolol—Beta-Blocker (BB)
Perindonril—Beta-Blocker (BB)
Pindolol—Beta-Blocker (BB)
Prazosin—Alpha-Blocker
Propranolol—Beta-Blocker (BB)
Quinapril—Angiotensin-Converting Enzyme Inhibitor (ACE-I)
Ramipril—Angiotensin-Converting Enzyme Inhibitor (ACE-I)
Reserpine—Alkaloid
Spironolactone—Aldosterone Antagonist/Potassium-Sparing Diuretic
Telmisartan—Angiotensin II Receptor Antagonists (ANG2)
Terazosin—Alpha-Blocker
Timolol—Beta-Blocker (BB)
Torsemide—Loop Diuretic
Trandolapril—Angiotensin-Converting Enzyme Inhibitor (ACE-I)
Trandolapril; Verapamil—Combination of Angiotensin-Converting Enzyme Inhibitor (ACE-I)/Calcium-Channel Blocker (CCB)
Triamterene—Potassium-Sparing Diuretic
Valsartan—Angiotensin II Receptor Antagonists (ANG2)
Verapamil—Calcium-Channel Blocker (CCB)
   List 2a. Available Prescription Diabetes Mellitus Medications with Mechanism Class:
Acarbose—Alpha-Glucosidase Inhibitor
Acetohexamide—Sulfonylurea
Bromocritine—Hormone Modifier
Canagliflozin; Metformin—Combination of Sodium-glucose cotransporter 2 Inhibitor/Biguanide
Canagliflozin—Sodium-glucose cotransporter 2 Inhibitor
Chloropropamide—Sulfonylurea
Colesevelam—Bile Acid Sequestrant
Dapagliflozin—Sodium-glucose cotransporter 2 Inhibitor
Dapagliflozin; Metformin—Combination of Sodium-glucose cotransporter 2 Inhibitor/Biguanide
Emapagliflozin; Metformin—Combination of Sodium-glucose cotransporter 2 Inhibitor/Biguanide
Emapagliflozin; Linagliptin—Combination of Sodium-glucose cotransporter 2 Inhibitor/Dipeptidyl Peptidase-4 Inhibitors
Emapagliflozin—Sodium-glucose cotransporter 2 Inhibitor
Exenatide—Incretin Mimetic
Glimepiride—Sulfonylurea
Glimepiride; Pioglitazone—Combination of Sulfonylurea/Thiazolidinedione
Glimepiride; Rosiglitazone—Combination of Sulfonylurea/Thiazolidinedione
Glipizide—Sulfonylurea
Glipizide; Metformin—Combination of Sulfonylurea/Biguanide
Glyburide—Sulfonylurea
Glyburide; Metformin—Combination of Sulfonylurea/Biguanide
Levocamitine—Glucose tolerance
Linagliptin—Dipeptidyl Peptidase-4 Inhibitors
Metformin—Biguanide
Metformin; Pioglitazone—Combination of Biguanide/Thiazolidinedione
Metformin; Repaglinide—Combination of Biguanide/Meglitinide
Metformin; Rosiglitazone—Combination of Biguanide/Thiazolidinedione
Metformin; Saxagliptin—Combination of Biguanide/Dipeptidyl Peptidase-4 Inhibitors
Metformin; Sitagliptin—Combination of Biguanide/Dipeptidyl Peptidase-4 Inhibitors
Miglitol—Alpha-Glucosidase Inhibitors
Nateglinide—Meglitinide
Pioglitazone—Thiazolidinedione
Pramlintide—Amylin analogs
Repaglinide—Meglitinide
Rosiglitazone—Thiazolidinedione
Saxagliptin—Dipeptidyl Peptidase-4 Inhibitors
Sitagliptin—Dipeptidyl Peptidase-4 Inhibitors
Tolazamide—Sulfonylureas
Tolbutamide—Sulfonylureas
   List 3. Available Prescription Hyperlipidemia Medications with Mechanism Class:
Amlodipine; Atorvastatin—Combination of Calcium-Channel Blocker (CCB)/HMG-CoA Reductase Inhibitor (Statin)
Aspirin, ASA; Pravastatin—Combination of Salicylate Platelet Inhibitor/HMG-CoA Reductase Inhibitor (Statin)
Atorvastatin—HMG-CoA Reductase Inhibitor (Statin)
Cerivastatin—HMG-CoA Reductase Inhibitor (Statin)
Cholestyramine—Bile Acid Sequestrant
Colsevelam—Bile Acid Sequestrant
Colestipol—Bile Acid Sequestrant
Ezetimibe—Cholesterol Absorption Inhibitor
) Ezetimibe; Simvastatin—Combination of Cholesterol Absorption Inhibitor/HMG-CoA Reductase Inhibitor (Statin)
Fenofibrate—Fibric Acid Derivatives
Fenofibric Acid—Fibric Acid Derivatives
Fluvastatin—HMG-CoA Reductase Inhibitor (Statin)
Gemfibrozil—Fibric Acid Derivatives
Lovastatin—HMG-CoA Reductase Inhibitor (Statin)
Lovastatin; Niacin—Combination of HMG-CoA Reductase Inhibitor (Statin)/Combination of Water-soluble Antilipemic Vitamin
Niacin; Simvastatin—Combination of Water-soluble Antilipemic Vitamin/HMG-CoA Reductase Inhibitor (Statin)
Omega-3-acid-ethyl ester—Fat-soluble Antilipemic
Pitavastatin—HMG-CoA Reductase Inhibitor (Statin)
Pravastatin—HMG-CoA Reductase Inhibitor (Statin)
Rosuvastatin—HMG-CoA Reductase Inhibitor (Statin)
Simvastatin—HMG-CoA Reductase Inhibitor (Statin)
   List 4. Available Depression Symptom Prescription Medications with Mechanism Class:
Amitriptyline—Tricyclic Antidepressant (TCA)
Amitriptyline; Chlordiazepoxide Combination of Benzodiazepine/Tricyclic Antidepressant (TCA)
Amoxapine—Heterocyclic Antidepressant
Aripiprazole—Atypical Antipsychotic
Bupropion—Multiple Mechanism Antidepressant
Citalopram—Selective Serotonin Reuptake inhibitor (SSRI)
Clomipramine—Tricyclic Antidepressant (TCA)
Desipramine—Tricyclic Antidepressant (TCA)
Desvenlafaxine—Serotonin-Norepinephrine Reuptake Inhibitor
Doxepin—Tricyclic Antidepressant (TCA)
Duloxetine—Serotonin-Norepinephrine Reuptake Inhibitor
Ephedrine—Adrenergic Agonists
Escitalopram—Selective Serotonin Reuptake inhibitor (SSRI)
Fluoxetine—Selective Serotonin Reuptake inhibitor (SSRI)
Fluoxetine; Olanzapine Combination of Selective Serotonin Reuptake inhibitor (SSRI)/Atypical Antipsychotic
Fluvoxamine—Selective Serotonin Reuptake Inhibitors (SSRIs)
Imipramine—Tricyclic Antidepressant (TCA)
Isocarboxazid—Monoamine Oxidase Inhibitor (MAOIs)
Maprotiline—Heterocyclic antidepressant
Methylphenidate—Adrenergic Agonist
Mirtazapine—Heterocyclic Antidepressant
Nefazodone—Phenylpiperazine Antidepressant
Nortriptyline—Tricyclic Antidepressant (TCA)
Paroxetine—Selective Serotonin Reuptake inhibitor (SSRI)
Phenelzine—Monoamine oxidase inhibitor (MAOIs)
Protriptyline—Tricyclic Antidepressant (TCA)
Quetiapine—Atypical Antipsychotic
Sertraline—Selective Serotonin Reuptake inhibitor (SSRI)
Tranylcypromine—Monoamine oxidase inhibitor (MAOIs)
Trazodone—Heterocyclic antidepressant
Trimipramine—Tricyclic Antidepressant (TCA)
Venlafaxine—Serotonin-Norepinephrine Reuptake Inhibitors
Vilazodone—Multiple Mechanism Antidepressant
   List 1b. Definitions of Hypertension Unique Mechanisms

### Used to Create “Syncpress” Teaching Example:

Beta-Blocker (BB)—this mechanism blocks the effects of the sympathetic nervous system by blocking the effects of neurotransmitters such as norepinephrine. This blockade will tend to dilate the vasculature and slow the heart rate. Some beta-blockers, such as carvedilol, also block effects on alpha receptors to additionally release tension on the vasculature system.

Angiotensin-Converting Enzyme Inhibitor (ACE-I)—this mechanism involves the prevention of a potent vasoconstrictor called angiotensin 2. There are a number of secondary mechanisms including mild diuresis. (note: newer renin inhibitors and angiotensin 2 inhibitors differ in the location for which this similar mechanism pathway is manipulated, so the use of such a combination additively, even added to an ACE-I may be considered duplicate therapy, unless a further algorithm calculation caveat proportioned these 3 subclasses as one unique mechanism. In other words, if this unique pathway mechanism was ⅙ of the cooperative combination, then this mechanism's ⅙ could be comprised of ^{1/18} ACE-I, ^{1/18} renin inhibitor & ^{1/18} angiotensin 2 inhibitor, with ^{1/18} being the fraction of the total cooperative combination system, and ⅙ being the additive total of the 3 distinct but less unique mechanisms of renin angiotensin system pathway).

Diuretic—this mechanism hinges upon the removal of sodium from plasma and extracellular fluid volume via the kidneys. The removal of sodium decreases the peripheral vascular resistance

Calcium-Channel Blocker (CCB)—this mechanism prevents the influx of extracellular calcium across the myocardial and vascular cell membranes without changing the plasma levels of calcium. This is another mechanism to decrease tension on the vasculature.

Centrally-Acting Adrenergic Agent—this mechanism involves agonist effects in the medulla, an effort that reduces the sympathetic response of the body. In simple terms, these medications prevent the release of norepinephrine and can reduce the effects of renin. Both chemicals add to the effects of hypertension.

Vasodilator—some of the total mechanisms are not totally understood, especially as it relates to hydralazine, but this mechanism is known to dilate arterioles more than the venous system when decreasing peripheral vascular resistance.

List 2b. Definitions of Diabetes Mellitus Unique Mechanisms

### Used to Create “Mellyces” Teaching Example:

Biguanide—this medication has multiple mechanisms to increase glucose tolerance: decrease glucose production in the liver, decreases the absorption of glucose in the small intestine and increase tissue sensitivity to insulin.

Dipeptidyl Peptidase-4 Inhibitors—this mechanism of glucose control increases insulin synthesis and decreases levels of glucagon, both leading to less sugar in the blood

Sulfonylureas—this mechanism stimulates insulin release from the pancreas to help

push sugar into the cells of the body

Thiazolidinedione—this mechanism increases the tissue sensitivity to insulin, including fat tissues, muscle tissues and the liver.

List 3b. Definitions of Hyperlipidemia Unique Mechanisms

### Used to Create “Eclepid” Teaching Example:

HMG-CoA Reductase Inhibitor (Statin)—this mechanism disrupts an enzyme used to make cholesterol in the liver and it also helps the body to get rid of the worst cholesterol

Fibric Acid Derivatives—this mechanism is not fully understood, but it inhibits the

formation of triglycerides and increases the breakdown of certain triglyceride lipoproteins

Cholesterol Absorption Inhibitor—prevents the absorption of cholesterol in the small

### Intestine

List 4b. Definition of Depression Symptom Alleviation Unique Mechanisms

### Used to Create “Lucidotion” Teaching Example:

Selective Serotonin Reuptake inhibitor (SSRI)— this mechanism is not fully understood, but it relates to potent inhibition of serotonin in the central nervous system and potentiating the effects of neurotransmissions associated with pleasure

Serotonin-Norepinephrine Reuptake Inhibitors—this mechanism relates to both inhibition of serotonin and norepinephrine, with serotonin inhibition often greater. This may also lead to inhibition of dopamine. All of these chemicals are associated with pleasure.

Heterocyclic Antidepressant—this mechanism is not fully understood, but depending on the dose, and even the particular heterocyclic compound, the mechanism relates to serotonin reuptake blocking in the presynaptic membrane. This may involve blocking presynaptic alpha-2 receptors to release serotonin. The release of norepinephrine can occur. Post synaptic serotonin receptors may be blocked and the subtypes of serotonin may differ.

Tricyclic Antidepressant (TCA)—the detailed mechanism is not fully understood, but it is thought that the most important effect is the decreased reuptake of norepinephrine and serotonin but do not affect dopamine reuptake

Adrenergic Agonist—this stimulant mechanism relates to a dopamine uptake blockade of central adrenergic neurons, likely near the brainstem and cerebral cortex. This is associated with pleasurable feelings.

Atypical Antipsychotic—the mechanism relates to manipulation of both dopamine and serotonin receptors, some have enhanced abilities to partially agonize activity at the D2 receptor since the medication can act as an antagonist at postsynaptic D2 receptors and a weak agonist at presynaptic D2 receptors. This is thought to be the case for aripiprazole.

List 1c. Conventional Cartoon Chemical Structures of Hypertension Medications

### Creating “Syncpress” Teaching Example:

### Carvedilol

C₂₄H₂₆N₂O₄

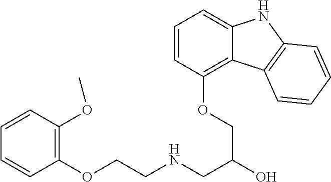 [SMILES: COc1ccccc1OCCNCC(O)COc1cccc2[nH]c3ccccc3c12]

### Lisinopril

C₂₁H₃₁N₃O₅.2H₂O

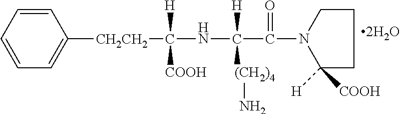

### Chlorthalidone

C₁₄H₁₁ClN₂O₄S

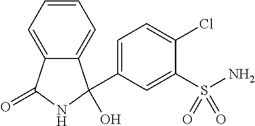 [SMILES: NS(=O)(=O)c1cc(C2(O)NC(=O)c3ccccc32)ccc1Cl]

### Amlodipine

C₂₀H₂₅CIN₂O₅

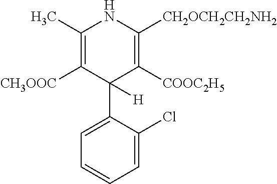

### Clonidine Hydrochloride

C₉H₉Cl₂N₃.HCl

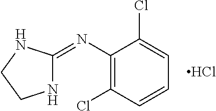 [SMILES: Clc1cccc(Cl)c1N=C1NCCN1 ; Cl]

### Hydralazine Hydrochloride

C₈H₈N₄.HCl

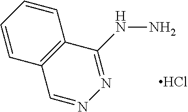 [SMILES: NNc1nncc2ccccc12 ; Cl]

List 2c. Conventional Cartoon Chemical Structures of Diabetes Mellitus Medications

### Creating “Mellyces” Teaching Example:

### Metformin

C₄H₁₁N₅

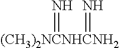

### Sitagliptin

C₁₆H₁₅F₆N₅O

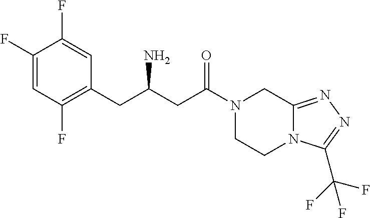 [SMILES: N[C@@H](CC(=O)N1CCn2c(nnc2C(F)(F)F)C1)Cc1cc(F)c(F)cc1F]

### Glyburide

C₂₃H₂₈ClN₃O₅S

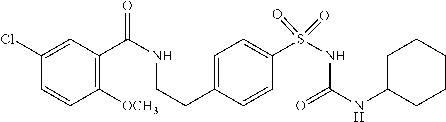 [SMILES: COc1ccc(Cl)cc1C(=O)NCCc1ccc(S(=O)(=O)NC(=O)NC2CCCCC2)cc1]

Pioglitazone hydrochloride
C₁₉H₂₀N₂O₃S.HCl

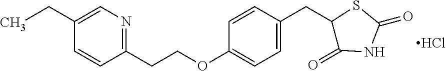 [SMILES: CCc1ccc(CCOc2ccc(CC3SC(=O)NC3=O)cc2)nc1 ; Cl]

### List 3. Conventional Cartoon Chemical Structures of Dyslipidemia Medications

### Creating “Eclepid” Teaching Example:

### Simvastin

C₂₅H₃₈O₅

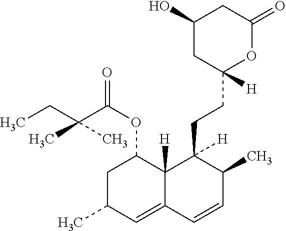

### Fenofibrate

C₂₀H₂₁O₄Cl

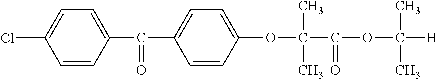 [SMILES: CC(C)OC(=O)C(C)(C)Oc1ccc(C(=O)c2ccc(Cl)cc2)cc1]

### Ezetimibe

C₂₄H₂₁F₂NO₃

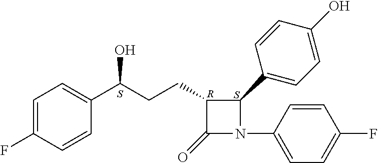

List 4c. Conventional Cartoon Chemical Structures of Depression Medications Creating

### “Lucidotion” Teaching

### Example:

### Sertraline

C₁₇H₁₈C₁₃N

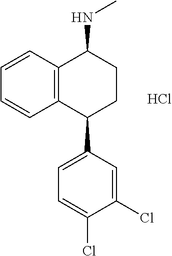 [SMILES: CN[C@H]1CC[C@@H](c2ccc(Cl)c(Cl)c2)c2ccccc21 ; Cl]

### Venlafaxine Hydrochloride

C₁₇H₂₇NO₂.HCl

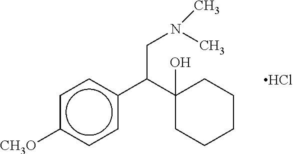

### Mirtazapine

C₁₇H₁₉N₃

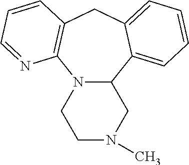 [SMILES: CN1CCN2c3ncccc3Cc3ccccc3C2C1]

### Trazodone Hydrochloride

C₁₉H₂₂CIN₅O.HCl

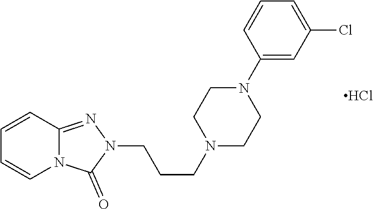 [SMILES: O=c1n(CCCN2CCN(c3cccc(Cl)c3)CC2)nc2ccccn12 ; Cl]

### Amitriptyline Hydrochloride

C₂₀H₂₃N.HCl

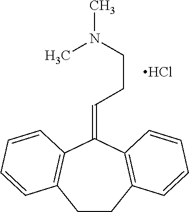 [SMILES: CN(C)CCC=C1c2ccccc2CCc2ccccc21 ; Cl]

### Aripiprazole

C₂₃H₂₇CL₂N₃O₂

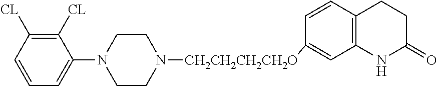

### DETAILED DESCRIPTION OF THE INVENTION

The present invention is a method for combining substandard doses to create pharmaceutical compositions to treat disease, the method comprising of the steps of an algorithm calculating a proportional substandard dose for each of at least three medications of varying mechanisms based on the total number of claimed at least three medications utilized, and combining claimed proportional substandard doses for claimed at least three medications to create claimed pharmaceutical composition, where each of the at least three claimed medications are further comprised of a unique mechanism treating the same disease, creating a final pharmaceutical composition that improves the treatment of claimed same disease.

Describing the method another way: step 1 requires selection from readily available standard grouping of pharmaceutical compositions that treat the same disease; step 2 requires a delineation of at least 3 unique pharmaceutical composition mechanisms for which the same disease is treated; step 3 requires the algorithm calculation of substandard doses for at least 3 pharmaceutical compositions of a unique mechanism treating the same disease; then step 4 is the final step of creating a final similar pharmaceutical composition, but this is now a combination of a series of substandardly dosed medications to treat the same disease via various cumulative mechanisms.

In addition to the at least three medications, the pharmaceutical composition may also further comprise any of the following additional components selected from the group consisting of vitamins, minerals, supplements (including supplements of human microbiota and foods), over the counter medications, off label prescriptions (legend medications for which formal approval of intended use was not granted), herbs and nutraceuticals.

The algorithm calculation of the new pharmaceutical composition would entail taking a substandard dosed medication from List Xa, using a unique mechanism defined per List Xb, defined as A(ss), and adding to another substandard dosed medication from List Xa, differing in mechanism defined per List Xb to the mechanism of chosen A(ss), defined as B(ss), and also minimally adding C(ss) from List Xa, where C(ss) differs in mechanism from both chosen A(ss) and also chosen B(ss) mechanisms and follows the species pattern for treating an indication of various conditions. For example, for treating an indication of hypertension, one would use List 1a and List 1b for the substandard dosed medications and the various mechanisms of each of the at least three medications and calculate the dosage of each medication as a proportion of the number of medications used in the newly combined pharmaceutical composition. If 4 medications are used, then the dosages would be approximately ¼ the standard low dosage. The substandard range is distinctly defined as the 90% quantity directly between a standard low dose and zero, as further described in the background.

The coordinated goal desired would be defined as an improved treatment effect and/or reduced side-effects of the standard dosage of the individual medications. Although not the initial step of the method, a crucial step is that of creating a pharmaceutical composition, because the intent of this combination method is to better treat disease with such a created composition.

Areas of the greatest concentration of medical resources, such as hypertension, diabetes mellitus, dyslipidemia and symptomatic treatment of depression can help to best illustrate implementation of the cooperative combination system method, from the extensive available research and development in these treatment areas. Each of these given diseases have numerous unique mechanisms for which pharmaceuticals are classified and used as standardized treatment. It would seem the most common diseases of greatest significance in the general population elude even the greatest attempts at controlling their pathologic progression with the current potent method. With this in noted, quite often multiple drug regimens are eventually required to lessen illness, but still consequent comorbidities and even direct mortality evade the best conventional attempts with the current pharmaceutical treatment approach.

Consider the numerous pharmaceutical chemical agents available to treat hypertension, including various dosage forms. There are slightly more than a half-dozen distinct physiologic mechanisms of action in this pharmacopoeia. Interestingly, there are nearly one hundred available hypertension medications (see List 1a). There are even potent combination medications, combining various mechanisms at no less than low standard doses. These combinations provide doses that are therapeutic individually, and are in one tablet/capsule, for advanced hypertension, much like the polypill combinations mentioned earlier. These combinations too are studied and approved compositions to be effective minimally at a given standard dose. It is crucial to note, whether it is a combination seen on the list provided or the polypill concept, these are not the same as calculating six unique, via mechanism of action, medications and decreasing the low dose proportionally, by about ⅛, to a substandard degree, for newly diagnosed uncomplicated hypertension. The combination of 6 chemical mechanisms, contoured to roughly ⅙ of a general low dose creates a composition that reinvents the current hypertension treatment standard. In other words, the doses detailed in this teaching have previously not been feasibly considered for practical use in the medical community, nor, more importantly, has it been feasible for them to be available in a pharmaceutical combination for wide scale disease based management.

However, it is well understood that many diseases, especially chronic ones, benefit from or eventually require more than one agent to more adequately mitigate the malady. M R Law and N J Wald, et. al. orchestrated a meta-analysis of 354 randomized trials to appreciate the value of low dose combinations of oral pharmaceuticals on blood pressure lowering. This low dose corresponds to the FDA designated minimally effective doses. The low dose is often less than the dose needed to obtain a typical target blood pressure in a typical hypertensive patient. This analysis was published by the British Medical Journal in 2003. The authors state no trial has studied the effect of three hypertension drugs in pharmaceutical combination, but it is suggested the effects would likewise be additive. The authors even report they have a patent application for a formula regarding a combination pill to reduce 4 cardiovascular risk factors, much like the polypill mentioned earlier.

It is rational to assume the novel method in this cooperative medication patent application has never occurred to the authors of the meta-analysis. If such a method did occur to the authors, then the authors would not limit the utility of combining to just 4 cardiovascular risk factors, certainly not to one inferior composition for such a limited group of patients. Nothing of the published meta-analysis suggests the authors uncovered the novelty of beyond low dose or substandard combination therapy, nor has another entity been known to teach such a rational utility of radical disease based improvements of current therapy.

The clinical ALLHAT trial illustrates only about one-third of hypertensive patients were treated effectively with a single potent medication agent. The 2014 Evidence-Based Guideline for the Management of High Blood Pressure in Adults Report From the Panel Members Appointed to the Eighth Joint National Committee (JNC 8) states advanced hypertension requires combination pharmaceutical therapy. There is much indecision regarding how to standardize the titration of combination therapy and what agents best serve the general population of hypertensive patients. This may well be related to the great concentration of single mechanism medications, used in high concentration, sought to mitigate complex pathologies, especially with the backdrop of significant malpractice litigation when decisions are made with excess ambiguity and poor outcomes result. It is rational for this patent's teaching to offer unexpected results when evaluating clinical data points and even ease practicality of implementation.

Carvedilol is a hypertension medication that blocks alpha-1 adrenergic receptors, and blocks both beta 1 and beta 2 adrenergic receptors. Lisinopril prevents angiotensin converting enzyme from converting angiotensin 1 to angiotensin 2 to lessen blood pressure. Chlorthalidone prevents reabsorption of sodium and chloride in the kidney, creating a diuretic effect. Amlodipine prevents calcium ion passage into vascular smooth muscle and the myocardium. Clonidine works in the central nervous system to block alpha-2 adrenergic receptors. Hydralazine dilates peripheral vessels directly. These classes of medications are not uncommonly used together at potent doses in the conventional model, namely with advanced disease. Potassium and magnesium are electrolytes lost when using chlorthalidone. Pyridoxine is a vitamin involved in the mechanism of hydralazine. Coenzyme Q10 is found in most cells in the body and its deficiency is associated with many maladies, including hypertension. Under the landmark combination method, one may use the algorithm to calculate carvedilol 1 mg (low dose=6.25 mg), lisinopril 2 mg (low dose=2.5 mg), chlorthalidone 4 mg (low dose=25 mg), amlodipine 0.8 mg (low dose=2.5 mg), clonidine 0.02 mg (low dose=0.2 mg), hydralazine 5 mg (low dose=40 mg), potassium 1 meq, pyridoxine 20 mg, magnesium 10 mg and Coenzyme Q10, and combine the substandard doses of raw materials in a single oral pharmaceutical composition. This algorithm driven composition would contain at least 3 substandard doses combined. There are at least 3 unique mechanisms with substandard doses via proportional calculation. All of the doses fall within the 90% substandard range, a range excluding doses greater than 95% below the low dose and further excluded from doses less than 5% of the low dose. This is a cooperative medication combination system, “Syncpress,” from Table 1. This tablet would provide more thorough treatment of the initial uncomplicated chronic hypertension process (See FIG. 1), and do it in a way that side-effects are mitigated (See FIG. 2).

The idea of coordinating the longer half-life medications, such as lisinopril and amlodipine to dissolve in an immediate release exterior of the tablet while executing a center matrix for slow-delivery of the remaining anti-hypertensives is a composition detail beyond the embodiment of this unique method (See FIG. 3). Either way, the process lends itself to easier dose titration and tapering, thereby further lessening side-effects via the various half lives of the various pharmaceutical medications. However, a biphasic tablet, possibly scored for further enhanced titration/tapering efforts, or the need to execute twice daily dosing, will need consideration. Also, determining the practical stability of such a combination of medications is beyond the teaching intent of this novel treatment practice. Further details, such as proportioning this prototype combination from a series of low starting doses or a consensus epidemiologic effective treatment dose is left for those with more medical actuary information, be it for initially treating a disease state of uncomplicated hypertension or any illness sought with this new method.

Likewise, type 2 diabetes mellitus (formerly “non-insulin dependent” or “adult onset” diabetes) is such a wide-scale malady that many pharmaceutical medications exist, including various dosage forms, with fewer unique mechanisms. However, a cooperative combination using the algorithm calculating a proportion of roughly ¼ the low dose or a consensus epidemiologic effective treatment dose would stand to allow the disease treatment to be effective longer, with greater tolerability, etc. The parts of a pharmaceutical composition combination include the raw ingredients: metformin, sitagliptin, glyburide and pioglitazone. Metformin decreases glucose production in the liver. It decreases the absorption of glucose in the small intestine and it increases tissue sensitivity to insulin. Sitagliptin increases insulin synthesis and decreases levels of glucagon. Glyburide stimulates insulin release from the pancreas. Pioglitazone increases insulin sensitivity in the tissues. These classes of medications are not uncommonly used together at potent doses in the conventional model, namely with advanced disease. Levocarnitine is associated with improved glucose utilization. Psyllium husk slows the absorption of glucose and aids in the prevention of diabetic gastropathy. Gut probiotics are associated with positive glucose management. Cinnamon is associated with glucose homeostasis. Resveratrol is a dietary antioxidant that has been associated with the prevention of heart disease, a pathology associated with diabetes. A metformin 200 mg (low dose=1000 mg), sitagliptin 12.5 mg (low dose=25 mg), glyburide 0.3 mg (low dose=2.5 mg), pioglitazone 4 mg (low dose=15 mg), levocarnitine 200 mg (low dose=990 mg), gut probiotics, cinnamon, with psyllium husk and resveratrol collaboration could be a revolution in diabetes treatment (See FIGS. 1 & 2). This algorithm driven composition would contain at least 3 substandard doses combined. There are at least 3 unique mechanisms with substandard doses via proportional calculation. This is a cooperative medication combination system, “Mellyces,” from Table 2.

The idea of coordinating the longer half-life medications to dissolve in an immediate release exterior of the tablet and executing a center matrix for slow-delivery of the metformin is a detail beyond the intent of this unique method (See FIG. 3). Either way, the process lends itself to easier titration and tapering, thereby further lessening side-effects via the various half lives of the various medications. However, a biphasic tablet, possibly scored for further enhanced titration/tapering efforts, or the need to offer twice daily dosing, will need consideration. The proportions may differ when clinical actuary or trial data is further developed, but the general method is rational, unique, and untried.

Diabetes Mellitus, like hypertension, is well understood to need combination pharmaceutical therapy (See examples in List 2a). It is common consumer knowledge. Consumer Report's Best Buy Drugs, updated December 2012, focused on oral Diabetes medications, clearly stating, on the recommendation page, that taking more than one diabetes drug is often necessary, but taking more than one diabetes drug raises the risk of adverse effects and increases costs. Cooperative combination medication systems makes the treatment need feasible while conserving resources.

Similarly, treating dyslipidemia would be best accomplished, initially, with a cooperative combination of medications. List 3a includes commercially available combinations that even include medications for different indications, such as dyslipidemia and hypertension, or hyperlipidemia and clot prevention. As noted, the polypills mentioned previously combine various hypertension medications with an anti-clot mechanism, a hyperlipidemia treatment, some compositions even containing potassium.

Simvastatin reduces 3-hydroxy-3-methylglutaryl-coenzyme A reductase in such a way that it eliminates much of the fatty substances associated with cholesterol disease. Fenofibrate works in a fashion not fully understood, but inhibits the formation of triglycerides and increases the breakdown of certain triglyceride lipoproteins. Ezetimibe prevents the absorption of cholesterol in the small intestine. Niacin works to decrease bad cholesterol made by the liver, inhibits fat tissue lipolysis, decreases liver esterification, and increases lipoprotein lipase activity. These classes of medications are not uncommonly used together at potent doses in the conventional model, namely with advanced disease. Coenzyme Q10 is found in most cells in the body and its deficiency is associated with many maladies. The use of a “statin” medication such as simvastatin reduces coenzyme Q10. Omega-3-acid ethyl esters are part of the general population's dietary deficiency; supplementation is associated with reduced liver triglyceride synthesis. Resveratrol is a dietary antioxidant that has been associated with the prevention of heart disease. Pyrroloquinoline quinone has antioxidant capability associated with reduced heart muscle stress. Acetylcysteine is related to amino acids and replaces glutathione stores and relates to positive cardiac effects. The combining of substandard algorithm calculated simvastatin 4 mg (low dose=5 mg), fenofibrate 5 mg (low dose=54 mg), ezetimibe 0.5 mg (only dose=10 mg) with niacin, coenzyme Q10, omega-3-acid ethyl esters, resveratrol, pyrroloquinoline quinone and acetylcysteine is consistent with the landmark process of this application. The idea of coordinating all but the simvastatin to be released immediately while executing a center matrix for evening delivery of simvastatin is a detail beyond the teaching embodiment of this unique method (See FIG. 3). This algorithm driven composition would contain at least 3 substandard doses combined. There are at least 3 unique mechanisms with substandard doses via proportional calculation. This is a cooperative medication combination system, “Eclepid,” from Table 3.

Another readily obvious cooperative combination, via the method, may even be applied to psychological illness. It is often seen in clinical practice, that like the abovementioned pharmaceutical combinations, the use of one available prescription medication is too often inadequate to appropriately control a given indication. Often times, this may be related to many complicated confounding factors, but in the case of treating psychological maladies, the placebo effect is much greater. It may be anticipated that a reinvention of the current model can amplify such an effect, but if the chemicals used to symptomatically treat such psychological manifestations offer relief individually, then again the pharmaceutical cooperative combination would be rational to hold more promise. A cooperative combination medication system would prevent the abrupt and seemingly inevitable need to add further pharmaceutical mechanisms of action.

Again, this indication offers standard commercially available pharmaceutical combinations (see List 4a). The combinations offered are for comorbidities of depression, and are done so with doses therapeutic for such comorbidity individually, comorbidities such as anxiety, bipolar symptoms, or psychosis. Likewise, the antidepressant dose in the commercial combination is also a therapeutic dose. These combinations on the list are not the cooperative substandard doses combined with at least three mechanisms used for the symptoms of depression treatment. It is not the standard of current practice to begin treatment with multiple mechanisms.

Sertraline works by selectively inhibiting the reuptake of serotonin, a chemical process associated with positive feelings. Venlafaxine inhibits the reuptake of norepinephrine, serotonin, and dopamine, multiple chemicals associated with positive feelings. Mirtazapine effects have not been fully elucidated, but have been associated with antagonizing alpha-2 adrenergic and serotonin 5-HT2 receptors. Similarly, trazodone effects have not been fully elucidated, but have been associated with antagonizing alpha-1 adrenergic and serotonin 5-HT2A and 5-HT2C receptors instead, while also inhibiting the reuptake of serotonin. Likewise, amitriptyline effects are not fully understood, but are associated with the inhibition of the reuptake of norepinephrine and serotonin. The full effects of methylphenidate are not fully known, but it is a central nervous stimulant that affects dopamine transport systems. Aripiprazole is also a medication with effects not fully understood, but it is known to partially agonize dopamine and serotonin 5-HT1A receptors while antagonizing serotonin 5-HT2A receptors. These classes of medications are not uncommonly used together at potent doses in the conventional model, namely with advanced disease. The effects of ergocalciferol are extensive but the association of positive feeling and the general dietary deficiency of the city-dwelling population are the aim of its addition. Similarly folic acid deficiency is associated with feelings of depression. Also, tryptophan is a dietary precursor to serotonin, a key chemical for feelings of pleasure. S-adenosylmethionine is produced in the body and supplementation has positive effects on depression symptoms. A proportional algorithm calculation can make a pharmaceutical composition treating the symptoms of depression as follows: Sertraline 4 mg (low dose=25 mg), venlafaxine 15 mg (low dose=75 mg), mirtazapine 2 mg (low dose=15 mg), trazodone 4 mg (low dose=25 mg), amitriptyline 4 mg (low dose=25 mg), methylphenidate 0.25 mg (low dose=2.5 mg), aripiprazole 0.25 mg (low dose=2 mg), with ergocalciferol, tryptophan, folic acid, and S-adenosylmethionine would be as comprehensive a chemical treatment there has ever been for the symptoms of depression, and consequently have the rational potential to be the safest and most effective at alleviating symptoms (See FIGS. 1 & 2). This algorithm driven composition would contain at least 3 substandard doses combined. There are at least 3 unique mechanisms with substandard doses via proportional calculation. This is a cooperative medication combination system, “Lucidotion,” from Table 4.

The process of coordinating the likely stimulating medications, such as sertraline, methylphenidate, and aripiprazole to dissolve in an immediate release exterior of a tablet, yet execute a center matrix for slow-delivery of the remaining likely sedatives, such as mirtazapine, trazodone and amitriptyline is a detail beyond the intent of teaching this unique idea (See FIG. 3). Either way, the method lends itself to easier titration and tapering, thereby further lessening side-effects via the various half lives of the various medications. However, a biphasic tablet, possibly scored for further enhanced titration/tapering efforts, or the need to offer twice daily dosing albeit with a non-uniform waking and also sleeping hour tablet, will need consideration.

The applications are many, and the abovementioned are species examples of this new art applied to make the rational art teaching tangible, to provide active steps to calculate and form a pharmaceutical composition. The prototype tablet proportions have been contoured to not be exact proportions, via the author's experiential knowledge in nearly 2 decades of intense study in the given areas of medicinal disease treatment. All proportions do fall within the defined range of a substandard dose.

As the potential to better mimic the reverse of a given pathology and/or consequently lessen side-effects that characterize a given treatment mechanism, the net intensity of medication interactions would likewise be lessened. Interactions will still be an issue, and in a greater quantity, as they are of clinical concern with the standard single potent active chemical agent model, but the clinical significance will reasonably be lessened when multiple mechanisms are represented in the way this art describes. The choice of which medication to prescribe for a particular indication, given hundreds of choices, will be mitigated through the radical simplification offered by cooperative combination therapy designed for complex disease. Initiating, increasing, or decreasing a given therapeutic regimen will be so rudimentary that medication errors will reasonably be significantly lessened, namely the Institute of Medicine reported most common errors.

In so doing, the comparative effects will be better understood with a larger population better treating complex disease similarly. Treating hypertension, diabetes, dyslipidemia, the symptoms of depression, chronic pain (methadone, tramadol, oxaprozin, gabapentin, acetaminophen, ashwagandha, and S-adenosylmethionine), osteoarthritis (oxaprozin, flavocoxid, prednisone, green lipped mussel, resveratrol, curcumin, acetaminophen, and pycnogenol), neuropathic pain (gabapentin, amitriptyline, duloxetine, baclofen, pyrroloquinoline quinone, and ashwagandha), seizure disorders (carbamazepine, lorazepam, lacosamide, gabapentin, levetiracetam, ashwagandha, magnesium and folic acid) constipation (linaclotide, lubiprostone, methylnaltrexone, polyethylene glycol, psyllium husk, docusate, and magnesium) insomnia (zolpidem, suvorexant, lorazepam, ramelteon, S-adenosylmethionine, and ashwagandha) hypertensive emergency/urgency (labetalol, enalaprilat, clonidine, and hydralazine), gastrointestinal erosion (lansoprazole, famotidine, misoprostol, iron, calcium acetate, arginine, glutamine, and methylcobalamin), glaucoma (latanoprost, dorzolamide, brimonidine, timolol, and acetylcysteine), allergy symptoms (loratadine, montelukast, prednisone, and green tea extract), fibromyalgia (duloxetine, milnacipran, amitriptyline, S-adenosylmethionine, green-lipped mussel, resveratrol, curcumin, and flavocoxid) schizophrenia (aripiprazole, molindone, quetiapine, hydroxytryptophan, ashwagandha), Parkinson's (carbidopa, levodopa, entacapone, ropinirole, pramipexole, selegiline, benztropine, pyrroloquinoline quinone, and ashwagandha), dementia (memantine, donepezil, rivastigmine, huperzine A, pyrroloquinoline quinone, panax ginseng, acetylcarnitine, and ashwagandha), emesis and nausea (aprepitant, ondansetron, prochlorperazine, trimethobenzamide, pyridoxine) etc., shows how practical application of the more abstract seeming similarity and consequent applied method illustrate the broad, yet defined, rational utility. The clinical inertia, such as which single choice is better, which choice to add to a failing regimen, or what side-effects are most problematic, are drastically reduced & clinical goals may be reached to a tremendous degree composing a cooperative medication algorithm-based system to treat disease.

This method can reasonably provide results otherwise unexpected. The use of pharmaceutical compositions to treat disease are currently expected to have a lesser efficacy and higher toxicity, certainly a lessened efficacy over extended periods of time. The method of cooperative medication combination systems can reasonably improve standard efficacy measures, lessen toxicity, and even extend the efficacy over extended periods.

Likewise, prescribers overwhelmed with patient workload are in need of a safer approach to achieve clinical goals when managing disease. Using the algorithm of cooperative medication combination systems can reasonably be used with an algorithm of surrogate endpoints for which patients can be better empowered to manage one's own care under the guidance of a licensed prescriber, detailed manufacturer resources, etc. Such unexpected results will dramatically improve the aspect of health care relating to pharmaceutical compositions.

### REFERENCES

Kubinyi, H. Drug research: myths, hype and reality. Nature Rev, Drug Discov. 2, 665-668 (2003).
Kitano, H. A robustness-based approach to systems-oriented drug design. Nature. 6, 202-210 (2007).
Kohn L T, Corrigan J M, Donaldson M S, editors. To err is human: building a safer health system. A report of the Committee on Quality of Healthcare in America, Institute of Medicine. Washington, D.C.: National Academy Press; 2000.
Thom S, Poulter N, Field J, Patel A, Prabhakaran D, Stanton A, Grobbee D E, Bots M L, Reddy K S, Cidambi R, Bompoint S, Billot L, Rodgers A; UMPIRE Collaborative Group. Effects of a fixed-dose combination strategy on adherence and risk factors in patients with or at high risk of CVD: the UMPIRE randomized clinical trial. JAMA. 2013 Sep. 4.
Indian Polycap Study (TIPS); Yusuf S, Pais P, Afzal R, Xavier D, Teo K, Eikelboom J, Sigamani A, Mohan V, Gupta R, Thomas N. Effects of a polypill (Polycap) on risk factors in middle-aged individuals without cardiovascular disease (TIPS): a phase II, double-blind, randomised trial. Lancet. 2009 Apr. 18; 373(9672):1341-51. Epub 2009 Mar. 30.
Comparison of risk factor reduction and tolerability of a full-dose polypill (with potassium) versus low-dose polypill (polycap) in individuals at high risk of cardiovascular diseases: the Second Indian Polycap Study (TIPS-2) investigators. Yusuf S, Pais P, Sigamani A, Xavier D, Afzal R, Gao P, Teo K K. Circ Cardiovasc Qual Outcomes. 2012 Jul. 1; 5(4):463-71. Epub 2012 Jul. 10.
Law M R, Wald N J, Morris J K, Jordan R E. Value of low dose combination treatment with blood pressure lowering drugs: analysis of 354 randomised trials. BMJ 2003; 326:1427-31
The ALLHAT Officers and Coordinators for the ALLHAT Collaborative Group. Major outcomes in high-risk hypertensive patients randomized to angiotensin converting enzyme inhibitor or calcium channel blocker vs diuretic. The Antihypertensive and Lipid-Lowering Treatment to Prevent Heart Attack Trial (ALLHAT). JAMA. 2002; 288:1981-1997.
Paul A. James, MD1; Suzanne Oparil, MD2; Barry L. Carter, PharmD1; et al. 2014 Evidence-Based Guideline for the Management of High Blood Pressure in Adults Report From the Panel Members Appointed to the Eighth Joint National Committee (JNC 8). JAMA. 2014; 311 (5):507-520. doi:10.1001/jama.2013.284427
Consumer Reports Health Best Buy Drugs, “The Oral Diabetes Drugs: Treating Type 2 Diabetes”, Best Buy Drugs (Consumer Reports), retrieved Sep. 18 2012
Clinical Pharmacology [database online]. Tampa, Fla.: Gold Standard, Inc.; 2006. URL: http://cp.gsm.com. Updated January 2011.

### FIG. 4. Algorithm Calculating the General Proportion of a Substandard Dose

$$Substandard Dose for A of Cooperative Medications Combination System = \frac{A}{n} = A ( ss )$$

$$Substandard Dose for B of Cooperative Medications Combination System = \frac{B}{n} = B ( ss )$$

$$Substandard Dose for C of Cooperative Medications Combination System = \frac{C}{n} = C ( ss )$$

The combination of A(ss), B(ss), and C(ss) into one pharmaceutical composition is equal to a Cooperative Medication Combination System.

1. A method for combining substandard doses to create pharmaceutical compositions to treat disease, the method comprising of the steps of an algorithm weighing a proportional substandard dose for each of at least three medications of varying mechanisms based on the total number of claimed at least three medications utilized, and combining claimed proportional substandard doses for claimed at least three medications to create claimed pharmaceutical composition, where each of the at least three claimed medications are further comprised of a unique mechanism treating the same disease, creating a final pharmaceutical composition that improves the treatment of claimed same disease

2. The method of claim 1, further comprised of a component selected from the group consisting of vitamins, minerals, supplements, over the counter medications, and/or herbs is/are added to claimed pharmaceutical combination of at least three medications.

6. The method of claim 1 wherein depression is treated by claimed combination of at least three medications, producing a pharmaceutical composition of sertraline 2 mg-20 mg, venlafaxine 5 mg-20 mg, amitriptyline 2 mg-8 mg, mirtazapine 1 mg-12 mg, trazodone 2 mg-20 mg, methylphenidate 0.25 mg-2 mg, and aripiprazole 0.25 mg-0.75 mg in a 24 hour dose.
